(19) United States
(12) Patent Application Publication
(10) Pub. No.: US 2026/0213010 A1
(43) Pub. Date: Jul. 23, 2026

(54) CONTROLLING POWER TO AN IMPLANTABLE DEVICE

(71) Applicant: Cochlear Limited, Macquarie University, NSW (AU)

(72) Inventors: Hans VANDENWIJNGAERDEN, West Ryde, NSW (AU); Helmut Christian EDER, Bemboka, NSW (AU)

(21) Appl. No.: 19/139,559

(22) PCT Filed: Dec. 15, 2023
(86) PCT No.: PCT/IB2023/062781

§ 371 Date: Jun. 16, 2025

Related U.S. Application Data
(60) Provisional application No. 63/476,698, filed on Dec. 22, 2022.

(51) Int. Cl. G16H 40/67; G05B 13/02; G05B 13/04; G06F 1/26

(52) U.S. Cl. CPC G16H 40/67; G05B 13/0265; G05B 13/048; G06F 1/266

(57) ABSTRACT

Presented herein are techniques for predicting and controlling power for an implantable device. The power prediction for the implantable device may be performed independent of real-time power information from the implantable device, and may utilize artificial intelligence (AI) or machine learning.

### BACKGROUND

### Field of the Invention

Aspects of the present invention relate generally to controlling power to an implantable device based on artificial intelligence (AI) or machine learning.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a method is provided. The method comprises: receiving environmental signals at an external device of an implantable medical device system; predicting, by a prediction model, a power level for an implantable medical device of the implantable medical device system to generate stimulation signals for delivery to a recipient of the implantable medical device system based on the environmental signals, wherein the prediction model predicts the power level based independent of real-time power information from the implantable medical device; and controlling power transmitted from the external device to the implantable medical device based on the predicted power level.

In another aspect, one or more non-transitory computer readable storage media comprising instructions are provided. The instructions, when executed by one or more processors, cause the one or more processors to: predict, by a prediction model based on audio signals, a power level for an implantable medical device to generate stimulation signals for delivery to a recipient, wherein the prediction model includes at least one machine learning model; and control power to the implantable medical device for generating the stimulation signals based on the predicted power level.

In another aspect, an external device of an implantable medical device system is provided. The external device comprises: memory for storing data; and one or more processors, wherein the one or more processors are configured to: predict, by a prediction model based on sensory signals, a power level for an implantable medical device to generate stimulation signals for delivery to a recipient, wherein the prediction model includes at least one machine learning model; and control power to the implantable medical device for the stimulation based on the predicted power level.

In another aspect, another method is provided. The method comprises: predicting, by a prediction model of an external device of an implantable medical device system based on sensory signals, a power level for an implantable medical device to generate stimulation signals for delivery to a recipient, wherein the prediction model includes at least one machine learning model; and controlling power from the external device to the implantable medical device based on the predicted power level.

### BRIEF DESCRIPTION OF THE DRAWINGS

Embodiments of the present invention are described herein in conjunction with the accompanying drawings, in which:

FIG. 1A is a schematic diagram illustrating a cochlear implant system with which aspects of the techniques presented herein can be implemented;

FIG. 1B is a side view of a recipient wearing a sound processing unit of the cochlear implant system of FIG. 1A;

FIG. 1C is a schematic view of components of the cochlear implant system of FIG. 1A;

FIG. 1D is a block diagram of the cochlear implant system of FIG. 1A;

FIG. 1E is a schematic diagram illustrating a computing device with which aspects of the techniques presented herein can be implemented;

FIG. 2 is a functional block diagram illustrating an example audio signal processing path of a cochlear implant system with which aspects of the techniques presented herein can be implemented;

FIG. 3 is a functional block diagram illustrating power level prediction and control for an implantable device according to certain techniques presented herein;

FIG. 4 is a graphical illustration of a load of an implantable device relative to predicted power provided to the implantable device and determined according to certain techniques presented herein;

FIG. 5 is a flowchart illustrating an example process to determine a power level for an implantable device based on audio signals according to certain embodiments;

FIG. 6 is an illustration of an example timing diagram for controlling power to an implantable device based on audio signals according to certain techniques presented herein;

FIG. 7 is a flowchart illustrating an example process to determine a power level for an implantable device based on data from an audio signal processing path according to certain embodiments;

FIG. 8 is an illustration of an example timing diagram for controlling power to an implantable device based on data from an audio signal processing path according to certain techniques presented herein;

FIG. 9 is a schematic diagram of an example neural network with which aspects of the techniques presented herein can be implemented;

FIG. 10 is a schematic diagram of an example machine learning decision tree with which aspects of the techniques presented herein can be implemented;

FIG. 11 is a flowchart illustrating an example process to determine a power level for an implantable device according to certain embodiments;

FIG. 12 is a flowchart illustrating another example process to determine a power level for an implantable device according to certain embodiments; and FIG. 13 is a schematic diagram illustrating a vestibular system with which aspects of the techniques presented herein can be implemented.

### DETAILED DESCRIPTION

Presented herein are techniques for predicting and controlling power for an implantable device. The power prediction may be performed at an external device, independent of real-time power information from the implantable device, and may use artificial intelligence (AI) or machine learning. A sound environment and other parameters associated with the implantable device and/or the external device are analyzed by a power level prediction model preferably employing machine learning to estimate power needs of the implantable device. This enables optimized power to be provided from the external device to the implantable device without relying on a backlink or telemetry providing feedback from the implantable device concerning the power needs (e.g., to avoid supplying too much or too little power, etc.). Stated differently, the power level prediction model accurately estimates or predicts power needs of the implantable device independent of feedback or information from the implantable device concerning the power needs.

Merely for ease of description, the techniques presented herein are primarily described with reference to a specific medical device system, namely a cochlear implant system. However, it is to be appreciated that the techniques presented herein can also be partially or fully implemented by other types of medical device systems. For example, the techniques presented herein can be implemented by hearing aid systems and/or auditory prosthesis systems that include one or more other types of auditory prostheses, middle ear auditory prostheses, bone conduction devices, direct acoustic stimulators, electro-acoustic prostheses, auditory brain stimulators, combinations or variations thereof, etc. The techniques presented herein can also be implemented in dedicated tinnitus therapy devices and tinnitus therapy device systems. In further embodiments, the techniques presented herein can also be implemented by, or used in conjunction with, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

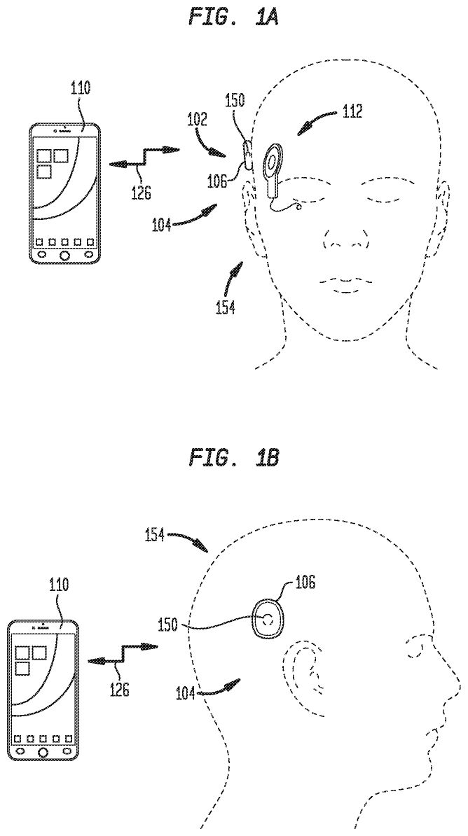

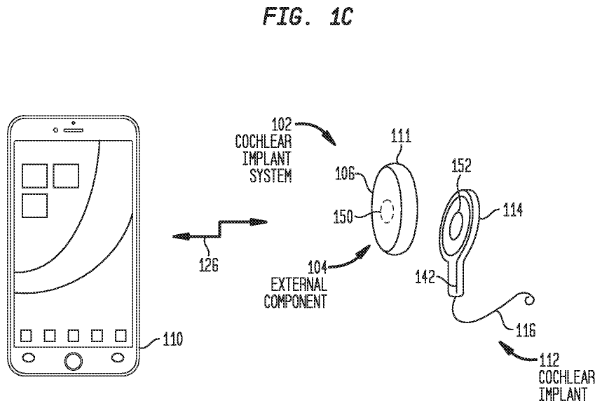

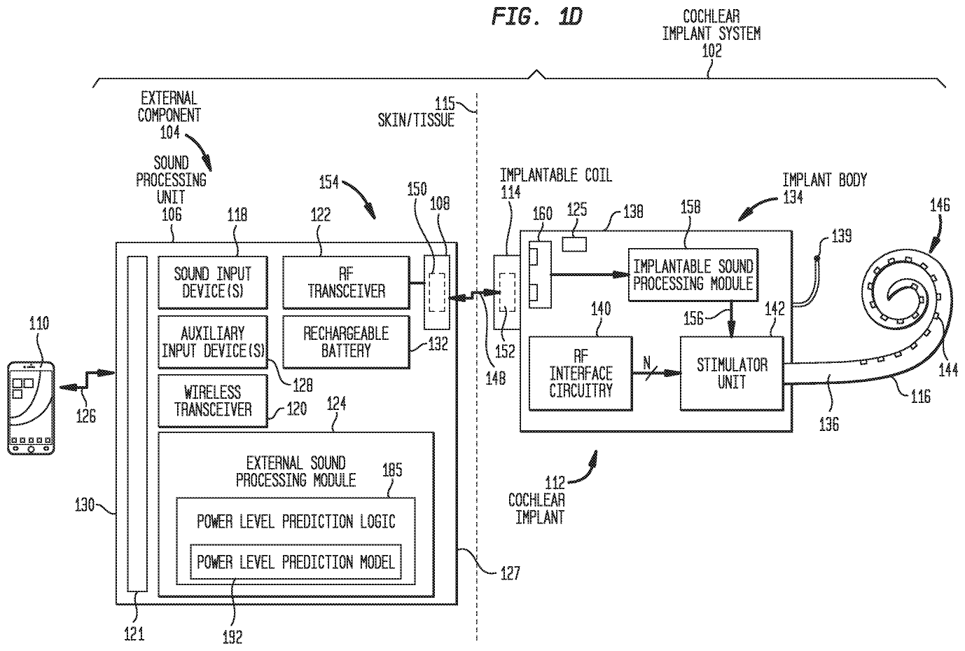

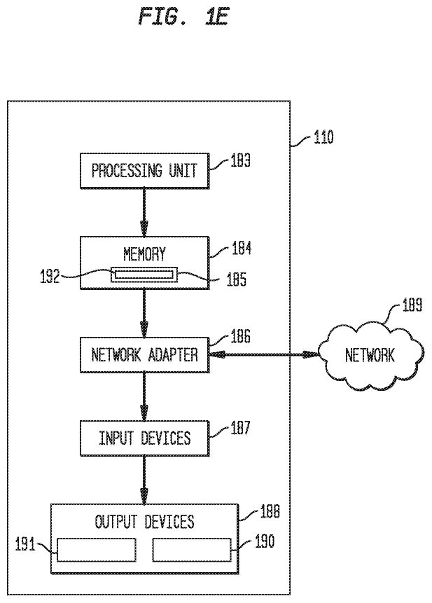

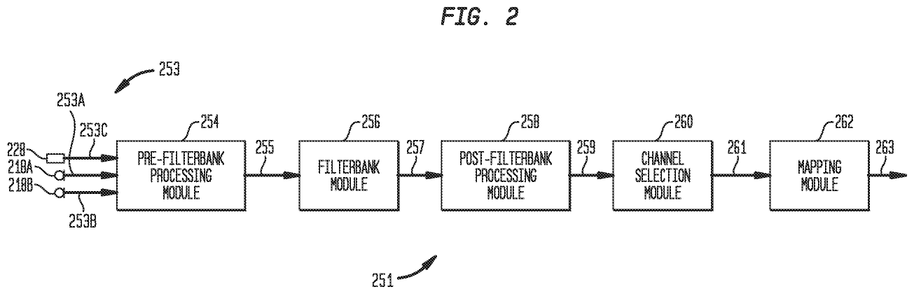

FIGS. 1A-1E illustrate an example cochlear implant system 102 with which aspects of the techniques presented herein can be implemented. The cochlear implant system 102 comprises an external component 104 and an implantable component 112. In the examples of FIGS. 1A-1E, the implantable component is sometimes referred to as a “cochlear implant.” FIG. 1A illustrates the cochlear implant 112 implanted in the head 154 of a user, while FIG. 1B is a schematic drawing of the external component 104 worn on the head 154 of the user. FIG. 1C is another schematic view of the cochlear implant system 102, while FIG. 1D illustrates further details of the cochlear implant system 102. For ease of description, FIGS. 1A-1E will generally be described together.

Cochlear implant system 102 includes an external component 104 that is configured to be directly or indirectly attached to the body of the user and an implantable component (or implant) 112 configured to be implanted in the user. In the examples of FIGS. 1A-1E, the external component 104 comprises a sound processing unit 106, while the cochlear implant 112 includes an implantable coil 114, an implant body 134, and an elongate stimulating assembly 116 configured to be implanted in the user's cochlea.

In the example of FIGS. 1A-1E, the sound processing unit 106 is an off-the-ear (OTE) sound processing unit, sometimes referred to herein as an OTE component, that is configured to send data and power to the implantable component 112. In general, an OTE sound processing unit is a component having a generally cylindrically shaped housing 111 and which is configured to be magnetically coupled to the user's head (e.g., includes an integrated external magnet 150 configured to be magnetically coupled to an implantable magnet 152 in the implantable component 112). The OTE sound processing unit 106 also includes an integrated (headpiece) coil 108 that is configured to be inductively coupled to the implantable coil 114.

It is to be appreciated that the OTE sound processing unit 106 is merely illustrative of the external devices that can operate with implantable component 112. For example, in alternative examples, the external component can comprise a behind-the-ear (BTE) sound processing unit or a micro-BTE sound processing unit and a separate external coil assembly. In general, a BTE sound processing unit comprises a housing that is shaped to be worn on the outer ear of the user and is connected to the separate external coil assembly via a cable, where the external coil assembly is configured to be magnetically and inductively coupled to the implantable coil 114. It is also to be appreciated that alternative external components can be located in the user's ear canal, worn on the body, etc.

As noted above, the cochlear implant system 102 includes the sound processing unit 106 and the cochlear implant 112. However, as described further below, the cochlear implant 112 can operate independently from the sound processing unit 106, for at least a period, to stimulate the user. For example, the cochlear implant 112 can operate in a first general mode, sometimes referred to as an “external hearing mode,” in which the sound processing unit 106 captures sound signals which are then used as the basis for delivering stimulation signals to the user. The cochlear implant 112 can also operate in a second general mode, sometimes referred as an “invisible hearing” mode, in which the sound processing unit 106 is unable to provide sound signals to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sound signals itself via implantable sound sensors and then uses those sound signals as the basis for delivering stimulation signals to the user. In certain examples, in the invisible hearing mode, an external device can still deliver power to the implant. In such examples, the external device can implement the techniques presented herein to use information (e.g., stimulation parameters) from the cochlear implant 112, retrieved or stored on the external device, to calculate an optimum power level. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 can also operate in alternative modes.

In FIGS. 1A and 1C, the cochlear implant system 102 is shown with an external computing device 110, configured to implement aspects of the techniques presented. The computing device 110, which is shown in greater detail in FIG. 1E, is, for example, a personal computer, server computer, hand-held device, laptop device, multiprocessor system, microprocessor-based system, programmable consumer electronic (e.g., smart phone), network PC, minicomputer, mainframe computer, tablet, remote control unit, distributed computing environment that include any of the above systems or devices, and the like. The computing device 110 can be a single virtual or physical device operating in a networked environment over communication links to one or more remote devices, such as an implantable medical device or implantable medical device system.

In its most basic configuration, computing device 110 includes at least one processing unit 183 and memory 184. The processing unit 183 includes one or more hardware or software processors (e.g., Central Processing Units) that can obtain and execute instructions. The processing unit 183 can communicate with and control the performance of other components of the computing device 110.

The memory 184 is one or more software or hardware-based computer-readable storage media operable to store information accessible by the processing unit 183. The memory 184 can store, among other things, instructions executable by the processing unit 183 to implement applications or cause performance of operations described herein, as well as other data. The memory 184 can be volatile memory (e.g., RAM), non-volatile memory (e.g., ROM), or combinations thereof. The memory 184 can include transitory memory or non-transitory memory. The memory 184 can also include one or more removable or non-removable storage devices. In examples, the memory 184 can include RAM, ROM, EEPROM (Electronically-Erasable Programmable Read-Only Memory), flash memory, optical disc storage, magnetic storage, solid state storage, or any other memory media usable to store information for later access. In examples, the memory 184 encompasses a modulated data signal (e.g., a signal that has one or more of its characteristics set or changed in such a manner as to encode information in the signal), such as a carrier wave or other transport mechanism and includes any information delivery media. By way of example, and not limitation, the memory 184 can include wired media such as a wired network or direct-wired connection, and wireless media such as acoustic, RF, infrared and other wireless media or combinations thereof. In certain embodiments, the memory 184 comprises power level prediction logic 185 that, when executed, enables the processing unit 183 to perform aspects of the techniques presented.

In the illustrated example, the computing device 110 further includes a network adapter 186, one or more input devices 187, and one or more output devices 188. The computing device 110 can include other components, such as a system bus, component interfaces, a graphics system, a power source (e.g., a battery), among other components.

The network adapter 186 is a component of the computing device 110 that provides network access (e.g., access to at least one network 189). The network adapter 186 can provide wired or wireless network access and can support one or more of a variety of communication technologies and protocols, such as ETHERNET, cellular, BLUETOOTH, near-field communication, and RF (Radiofrequency), among others. The network adapter 186 can include one or more antennas and associated components configured for wireless communication according to one or more wireless communication technologies and protocols. In certain examples, the one or more antennas can be shared with the charging coil 121 and/or external coil 108.

The one or more input devices 187 are devices over which the computing device 110 receives input from a user. The one or more input devices 187 can include physically-actuatable user-interface elements (e.g., buttons, switches, or dials), touch screens, keyboards, mice, pens, and voice input devices, among others input devices.

The one or more output devices 188 are devices by which the computing device 110 is able to provide output to a user. The output devices 188 can include, a display 190 and one or more speakers 191, among other output devices.

It is to be appreciated that the arrangement for computing device or system 110 shown in FIG. 1E is merely illustrative and that aspects of the techniques presented herein can be implemented at a number of different types of systems/devices. For example, the computing device 110 can be a laptop computer, tablet computer, mobile phone, surgical system, etc.

The OTE sound processing unit 106 comprises one or more input devices that are configured to receive input signals (e.g., sound or data signals). The one or more input devices include one or more sound input devices 118 (e.g., one or more external microphones, audio input ports, telecoils, etc.), one or more auxiliary input devices 128 (e.g., audio ports, such as a Direct Audio Input (DAI), data ports, such as a Universal Serial Bus (USB) port, cable port, etc.), and a wireless transmitter/receiver (transceiver) 120 (e.g., for communication with the external computing device 110). However, it is to be appreciated that one or more input devices can include additional types of input devices and/or less input devices (e.g., the wireless short range radio transceiver 120 and/or one or more auxiliary input devices 128 can be omitted).

The OTE sound processing unit 106 also comprises the external coil 108, a charging coil 121, a closely-coupled transmitter/receiver (RF transceiver) 122, sometimes referred to as radio-frequency (RF) transceiver 122, at least one rechargeable battery 132, and an external sound processing module 124. The external sound processing module 124 can comprise, for example, one or more processors and a memory device (memory) that includes sound processing logic. The memory device may further include power level prediction logic 185 that, when executed, enables the one or more processors to perform aspects of the techniques presented. The memory device can comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic and power level prediction logic 185 stored in memory device.

The implantable component 112 comprises an implant body (main module) 134, a lead region 136, and the intra-cochlear stimulating assembly 116, all configured to be implanted under the skin/tissue (tissue) 115 of the user. The implant body 134 generally comprises a hermetically-sealed housing 138 in which could potentially include at least one battery 125, RF interface circuitry 140, and a stimulator unit 142 are disposed. The implant body 134 also includes the internal/implantable coil 114 that is generally external to the housing 138, but which is connected to the RF interface circuitry 140 via a hermetic feedthrough (not shown in FIG. 1D).

As noted, stimulating assembly 116 is configured to be at least partially implanted in the user's cochlea. Stimulating assembly 116 includes a plurality of longitudinally spaced intra-cochlear electrical stimulating contacts (electrodes) 144 that collectively form a contact or electrode array 146 for delivery of electrical stimulation (current) to the user's cochlea.

Stimulating assembly 116 extends through an opening in the user's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external coil 108 and the implantable coil 114. The external magnet 150 is fixed relative to the external coil 108 and the implantable magnet 152 is fixed relative to the implantable coil 114. The magnets fixed relative to the external coil 108 and the implantable coil 114 facilitate the operational alignment of the external coil 108 with the implantable coil 114. This operational alignment of the coils enables the external component 104 to transmit power, and optionally data, to the implantable component 112 via a closely-coupled wireless link 148 formed between the external coil 108 with the implantable coil 114. In certain examples, the closely-coupled wireless link 148 is a radio frequency (RF) link. However, various other types of energy transfer, such as infrared (IR), electromagnetic, capacitive and inductive transfer, can be used to transfer the power and/or data from an external component to an implantable component and, as such, FIG. 1D illustrates only one example arrangement.

As noted above, sound processing unit 106 includes the external sound processing module 124. The external sound processing module 124 is configured to convert received input signals (received at one or more of the input devices) into output signals for use in stimulating a first ear of a user (i.e., the external sound processing module 124 is configured to perform sound processing on input signals received at the sound processing unit 106). Stated differently, the one or more processors in the external sound processing module 124 are configured to execute sound processing logic in memory to convert the received input signals into output signals that represent electrical stimulation for delivery to the user. The external sound processing module 124 may further estimate power needs of the implant 112 preferably using artificial intelligence (AI) or machine learning and provide power to the implant 112 based on the estimated power needs according to techniques presented herein.

As noted, FIG. 1D illustrates an embodiment in which the external sound processing module 124 in the sound processing unit 106 generates the output signals. In an alternative embodiment, the sound processing unit 106 can send less processed information (e.g., audio data) to the implantable component 112 and the sound processing operations (e.g., conversion of sounds to output signals) can be performed by a processor within the implantable component 112.

Returning to the specific example of FIG. 1D, the output signals are provided to the RF transceiver 122, which transcutaneously transfers the output signals (e.g., in an encoded manner) to the implantable component 112 via external coil 108 and implantable coil 114. That is, the output signals are received at the RF interface circuitry 140 via implantable coil 114 and provided to the stimulator unit 142. The stimulator unit 142 is configured to utilize the output signals to generate electrical stimulation signals (e.g., current signals) for delivery to the user's cochlea. In this way, cochlear implant system 102 electrically stimulates the user's auditory nerve cells, bypassing absent or defective hair cells that normally transduce acoustic vibrations into neural activity, in a manner that causes the user to perceive one or more components of the received sound signals.

As detailed above, in the external hearing mode, the cochlear implant 112 receives processed sound signals from the sound processing unit 106. However, in the invisible hearing mode, the cochlear implant 112 is configured to capture and process sound signals for use in electrically stimulating the user's auditory nerve cells. In particular, as shown in FIG. 1D, the cochlear implant 112 includes a plurality of implantable sound sensors 160 and an implantable sound processing module 158. Similar to the external sound processing module 124, the implantable sound processing module 158 can comprise, for example, one or more processors and a memory device (memory) that includes sound processing logic. The memory device can comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in the memory device.

In the invisible hearing mode, the implantable sound sensors 160 are configured to detect/capture signals (e.g., acoustic sound signals, vibrations, etc.), which are provided to the implantable sound processing module 158. The implantable sound processing module 158 is configured to convert received input signals (received at one or more of the implantable sound sensors 160) into output signals for use in stimulating the first ear of a user (i.e., the processing module 158 is configured to perform sound processing operations). Stated differently, the one or more processors in implantable sound processing module 158 are configured to execute sound processing logic in memory to convert the received input signals into output signals 156 that are provided to the stimulator unit 142. The stimulator unit 142 is configured to utilize the output signals 156 to generate electrical stimulation signals (e.g., current signals) for delivery to the user's cochlea, thereby bypassing the absent or defective hair cells that normally transduce acoustic vibrations into neural activity.

It is to be appreciated that the above description of the so-called external hearing mode and the so-called invisible hearing mode are merely illustrative and that the cochlear implant system 102 can operate differently in different embodiments. For example, in one alternative implementation of the external hearing mode, the cochlear implant 112 can use signals captured by the sound input devices 118 and the implantable sound sensors 160 in generating stimulation signals for delivery to the user.

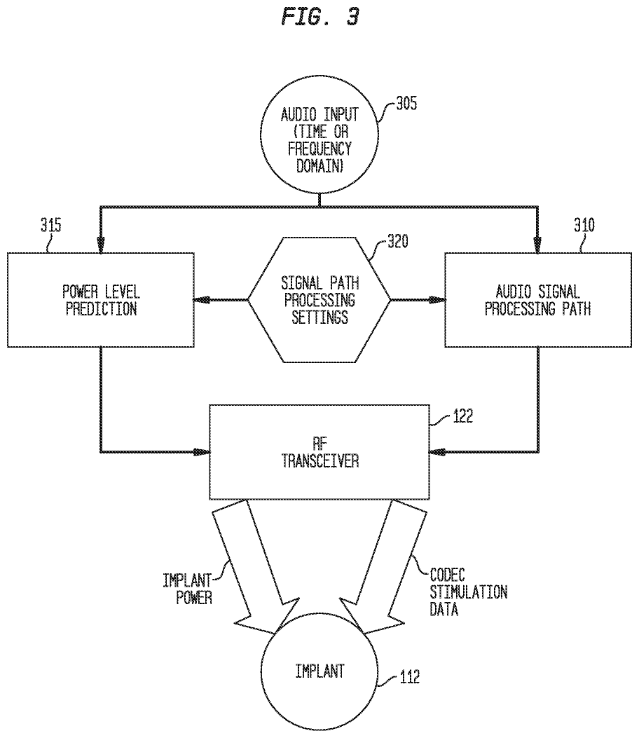

In at least one embodiment during operation of a hearing device system including a cochlear implant, as discussed in further detail below with reference to FIG. 2, sound processing module 124 is configured to convert output signals received from the input devices (e.g., one or more sound input devices 118 and/or one or more auxiliary input devices 128) into a set of output signals representative of electrical stimulation.

With reference to FIG. 2, shown is a functional block diagram illustrating an example sound/audio signal processing path of an auditory prosthesis, such as cochlear implant system 102, with which aspects of the techniques presented herein can be implemented. Various sound processing operations discussed for FIG. 2 can be performed via sound processing logic provided for any combination of an external component or an internal component of a cochlear implant system. Various features of cochlear implant system 102 as noted for FIGS. 1A-1D are discussed with reference to various features illustrated in FIG. 2.

Consider, with reference to FIG. 2, a sensory/environmental signal or audio signal processing path 251 which can be provided via sound processing module 124 of external component 104 and/or via sound processing module 158 of implantable component 112. In the example of FIG. 2, input devices can include two sound input devices, namely a first microphone 218A and a second microphone 218B, as well as at least one auxiliary input device 228 (e.g., an audio input port, a cable port, a telecoil, etc.). If not already in an electrical form, the input devices can convert received/input sound signals into electrical signals 253, referred to herein as electrical sound or sensory signals, which represent the sound/sensory signals received at the input devices. The electrical sound/sensory signals 253 can include electrical sensory signal 253A from microphone 218A, electrical sensory signal 253B from microphone 218B, and electrical sensory signal 253C from auxiliary input 228.

In FIG. 2, functional operations enabled by the audio signal processing path (i.e., the operations of one or more processor(s) when executing sound processing logic) are generally represented by modules 254, 256, 258, 260, and 262 which collectively comprise the audio signal processing path 251. Thus, the audio signal processing path 251 can include a pre-filterbank processing module 254, a filterbank module 256, a post-filterbank processing module 258, a channel selection module 260, and a mapping module 262, each of which are described in greater detail below.

Consider an operational example in which electrical sound signals 253 generated by the input devices are provided to the pre-filterbank processing module 254. The pre-filterbank processing module 254 is configured to, as needed, combine the electrical sound signals 253 received from the input devices and prepare/enhance those signals for subsequent processing. The operations performed by the pre-filterbank processing module 254 can include, for example, microphone directionality operations, noise reduction operations, input mixing/combining operations, input selection/reduction operations, dynamic range control operations and/or other types of signal enhancement operations. The operations at the pre-filterbank processing module 254 generate a pre-filterbank output signal 255 that, as described further below, is the basis of further sound processing operations. The pre-filterbank output signal 255 represents the combination (e.g., mixed, selected, etc.) of the input signals (e.g., mixed, selected, etc.) received at the sound input devices at a given point in time.

In operation, the pre-filterbank output signal 255 generated by the pre-filterbank processing module 254 is provided to the filterbank module 256. The filterbank module 256 generates a suitable set of bandwidth limited channels, or frequency bins, that each includes a spectral component of the received sound/sensory signals. That is, the filterbank module 256 comprises a plurality of band-pass filters that separate the pre-filterbank output signal 255 into multiple components/channels, each one carrying a frequency sub-band of the original signal (i.e., frequency components of the received sound/sensory signal).

The channels created by the filterbank module 256 are sometimes referred to herein as sound processing, or band-pass filtered, channels, and the sound signal components within each of the sound processing channels are sometimes referred to herein as band-pass filtered signals or channelized signals. The band-pass filtered or channelized signals created by the filterbank module 256 are processed (e.g., modified/adjusted) as they pass through the audio signal processing path 251. As such, the band-pass filtered or channelized signals are referred to differently at different stages of the audio signal processing path 251. However, it will be appreciated that reference herein to a band-pass filtered signal or a channelized signal can refer to the spectral component of the received sound signals at any point within the audio signal processing path 251 (e.g., pre-processed, processed, selected, etc.).

At the output of the filterbank module 256, the channelized signals are initially referred to herein as pre-processed signals or filterbank channels 257. The number ‘n’ of filterbank channels 257 generated by the filterbank module 256 can depend on a number of different factors including, but not limited to, implant design, number of active electrodes, coding strategy, and/or recipient preference(s). In certain arrangements, twenty-two (22) channelized signals are created and the audio signal processing path 251 is said to include 22 channels.

The filterbank channels 257 are provided to the post-filterbank processing module 258. The post-filterbank processing module 258 is configured to perform a number of sound processing operations on the target filterbank channels 257. These sound processing operations include, for example, channelized gain adjustments (e.g., performed via Loudness Growth Function (LGF) processing) for hearing loss compensation (e.g., gain adjustments to one or more discrete frequency ranges of the sound signals, also referred to herein as filter channels), noise reduction operations, speech enhancement operations, etc., in one or more of the channels. After performing the sound processing operations, the post-filterbank processing module 258 outputs a plurality of processed channelized signals 259.

In the specific arrangement of FIG. 2, the audio signal processing path 251 includes a channel selection module 260. The channel selection module 260 is configured to perform a channel selection process to select, according to one or more selection rules, which of the ‘n’ channels should be used in hearing compensation. The signals selected at channel selection module 260 are represented in FIG. 2 by arrow 261 and are referred to herein as selected channelized signals or, more simply, selected signals.

In the embodiment of FIG. 2, the channel selection module 260 selects a subset ‘m’ of the ‘n’ processed channelized signals 259 for use in generation of electrical stimulation for delivery to a recipient (i.e., the sound processing channels are reduced from ‘n’ channels to ‘m’ channels). In one specific example, the ‘m’ largest amplitude channels (maxima) from the ‘n’ available combined channel signals are made, with ‘n’ and ‘m’ being programmable during initial fitting, and/or operation of the prosthesis. In one instance, this specific example can be associated with an Advanced Combination Encoder (ACE), generally, a stimulation coding strategy, such as Optimized Pitch and Language (OPAL). It is to be appreciated that different channel selection methods could be used, and are not limited to maxima selection. It is also to be appreciated that, in certain embodiments, the channel selection module 260 can be omitted. For example, certain arrangements can use a continuous interleaved sampling (CIS), CIS-based, or other non-channel selection sound coding strategy.

The audio signal processing path 251 for the instance illustrated in FIG. 2 also includes the mapping module 262, which can generate output signals 263. In one embodiment, the mapping module 262 can be configured to map the amplitudes of the selected signals 261 (or the processed channelized signals 259 in embodiments that do not include channel selection) such that the output signals 263 correspond to a set of stimulation control signals (e.g., stimulation commands) that represent the attributes of the electrical stimulation signals that are to be delivered to a recipient so as to evoke perception of at least a portion of the received sound signals. This channel mapping can include, for example, threshold and comfort level mapping, dynamic range adjustments (e.g., compression), volume adjustments, etc., and can encompass selection of various sequential and/or simultaneous stimulation strategies.

In one embodiment, the set of stimulation control signals (stimulation commands) 263 that represent the electrical stimulation signals can be encoded for transcutaneous transmission (e.g., via an RF link) to an implantable component. As such, mapping module 262 can also be referred to as a channel mapping and encoding module and operates as an output block configured to convert the plurality of channelized signals into a plurality of stimulation control signals, from which the implantable component, via stimulator unit 142 can generate stimulation (current) signals for delivery to the recipient via a stimulating assembly 116.

In one embodiment, for example if channel selection module 260 is omitted from the audio signal processing path 251, the mapping module 262 can perform mapping operations that involve mapping channel envelopes to current levels, which can be mixed with streams received from one or more sources. Generally, a channel envelope is a “temporal envelope” that is extracted from each frequency band (channel) and is used to modulate pulse trains that are delivered to an implanted electrode. Thus, amplitudes of the current pulses can be extracted from the channel envelopes, where the channel envelopes correspond to the amplitude of the signal in a given frequency channel.

Thus, the audio signal processing path 251 generally operates to convert received sound signals into output signals 263, which can be used for delivering stimulation to a recipient in a manner that evokes perception of the sound signals.

As noted, implantable medical devices, such as cochlear implant 102, typically rely on power from one or more external devices for continued operation. This power is typically transferred via an inductive RF power link (e.g., wireless link 148). The power needs (or load) of the implantable medical device can vary significantly depending on many factors, such as sound environment, stimulation parameters, recipient impedances, etc. Accordingly, the power that an external device is sending to the implantable medical device should be optimized, since any surplus energy would be wasted and must be dissipated as heat. This additionally leads to shorter system battery autonomy.

Traditionally, implant systems attempt to estimate the power that needs to be transmitted from an external device to an implantable medical device by measuring the power needs on the implantable medical device. However, sending this information from the implantable medical device back over a communication link to the external device may be unreliable or incur an unacceptably high latency. This can prevent an implant system from reacting quickly enough with respect to the power needs of the implantable medical device, thereby causing the implantable medical device to become out of compliance and create a sub-optimal sound experience for a user. Alternatively, the implantable medical device may lose power completely and reset itself which creates an unpleasant experience for a user due to a short battery life or stimulation distortions and/or dropouts.

According to example embodiments, an external device leverages artificial intelligence (AI) or machine learning to accurately determine power needs for an implantable medical device for any given moment in time. The external device employs a power level prediction model that may include a variety of different machine learning models (e.g., neural network, machine learning decision tree, etc.) to determine the power needs. Simulations and various machine learning techniques are applied to train the power level prediction model for a wide variety of environmental/sensory input parameters and recipient impedance data models. Since the power level prediction model on the external device may exactly determine correct power needs of the implantable medical device for a given sound environment and impedance signature of a recipient, the external device does not need to rely on a backlink feedback loop from the implantable medical device to determine the power needs during operation (e.g., during processing of audio signals to provide stimulation, etc.). However, information from the implantable medical device may be used to train or update the power level prediction model. Further, an implant system can be implemented with greater power efficiency and sound perception performance. Moreover, since there is no requirement placed on the implantable medical device (e.g., to measure and provide power information, etc.) for power determination during operation, the machine learning power determination of example embodiments can be applied to any generation of implant technology.

In some instances, the power level prediction model is modelled around the characteristics of an implant system. The power level prediction model considers system specific parameters for determining power needs of an implant, such as wireless or RF link power transfer efficiency, implant system power model characteristics, etc. The power level prediction model may also consider a cochlea physiological model of a manner in which stimulation is absorbed and an impact of impedances measured at electrodes of the implant. The power level prediction model can be trained through simulation with a very wide data set of an audio sound environment detected by microphones, signal processing path parameters, and/or recipient impedance data. The power level prediction model may also have a training mode where data from an implant is fed back to the external device to further optimize and enhance the power level prediction model.

In some instances, a set of input parameters may be selected for the power level prediction model, such as an environment (e.g., quiet or loud environment, etc.) and/or other audio characteristics (e.g., signal strength, etc.) from an audio or environment classifier, filter bank channel weights, microphone sensitivity, etc. In addition, parameters from a currently loaded user MAP (e.g., maxima, comfort and threshold (C and T) levels, etc.) and/or other audio signal processing path settings may be used. During a fitting session (or application-controlled training mode), measured implant electrode impedances may be programmed into the power level prediction model. The parameters together with microphone data are used to accurately determine power needs of an implant system for a specific stimulation data set at a given moment in time. The determined power is applied in the implant system so that when stimulation is outputted on the electrodes, the external device delivers power matched to the power required to produce the stimulation within compliance limits of the implant system. This enables the external device to limit the amount of excessive power that is sent to the implant and extend the system battery autonomy.

For example, when a user walks from a quiet area into a noisy area, the environment from the environment or audio classifier changes and more output channels from the filter bank have an increased sound level. This is detected by the power level prediction model which determines that an electrical stimulation load will increase. Based on this new information, a new power need can be calculated which correctly increases power settings. The new power settings increase the power delivered to the implant just enough to compensate for the increased power load caused by the increase in the stimulation load or pulses.

By way of further example, when a recipient is walking next to a road and a loud truck suddenly passes, several input parameters (e.g., sound pressure levels from the filter bank, etc.) shortly increase and subsequently decrease as the truck moves away. The power level prediction model can react very quickly to these changes and momentarily increase the power delivered to the implant. The power delivered accurately matches the needs of the increased stimulation generated by the noise from the passing truck.

Present invention embodiments provide longer system battery autonomy by only sending the right amount of power to an implant at any given moment in time. Also, a recipient has a better sound perception/experience because stimulation always occurs with an acceptable out of compliance range. These advantages may be achieved without reliance on a potentially unreliable and often slow data back link from the implant to determine power needs during operation.

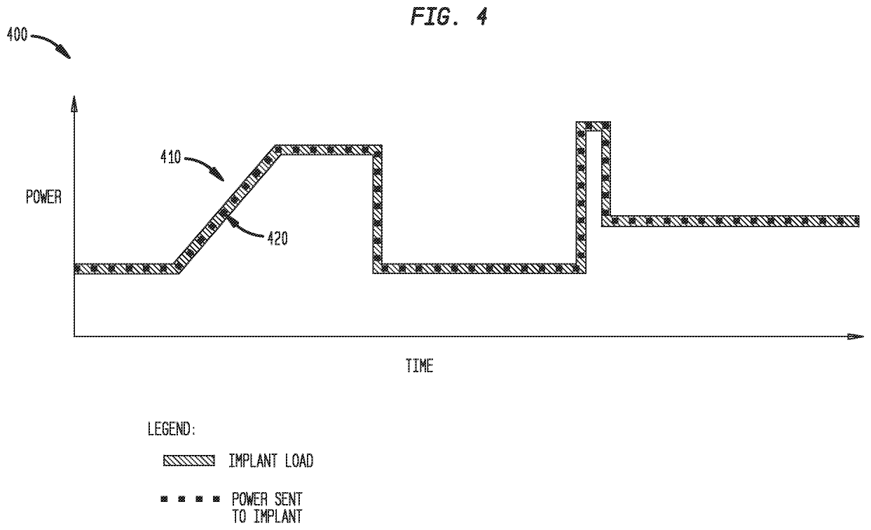

With reference to FIG. 3, shown is a functional block diagram illustrating power level prediction and control for an implantable device or implant (e.g., implant 112, etc.) according to certain techniques presented herein. By way of example, sound processing unit 106 of external component 104 receives audio input (or signals) that may be in the time or frequency domain at operation 305. The audio input may be received from microphone 218A, 218B, and/or at least one auxiliary input device 228 (FIG. 2). The audio signals are provided for the audio signal processing path at operation 310. In addition, settings are also provided at operation 320 for the audio signal processing path. The audio signal processing path may correspond to the audio signal processing path 251 described above for FIG. 2, and generally operates to convert received sound signals into output signals which can be used for delivering stimulation to a recipient in a manner that evokes perception of the sound signals. The settings are used for configuring the audio signal processing path.

The audio signals are further provided for power level prediction at operation 315. In addition, the settings for the signal processing path are also provided at operation 320 for the power level prediction. The power level prediction may be performed by power level prediction logic 185 (e.g., of external component 104, computing device 110, etc.) that utilizes power level prediction model 192 preferably employing artificial intelligence (AI) or machine learning to determine power needs of an implant (e.g., implant 112, etc.) based on one or more of various parameters as described below (e.g., audio input (e.g., in the time or frequency domain) or stimulation pulses, electrical current of each stimulation pulse, power efficiency coefficients for the implant, signal processing path settings (e.g., coding type, maxima, comfort and threshold (C and T) levels, stimulation rate, etc.), audio classification (e.g., quiet or loud environment, audio characteristics, etc.) from an environment or audio classifier, signal processing path information (e.g., filter bins or banks, signal strength levels, stimulation pulses, etc.), RF link settings and/or characteristics (e.g., frame period, frame occupation, power transfer coefficient, amount of signal being enabled/transferred (e.g., more power may be transferred as more data is sent), etc.), cochlea impedance measurements, implant (hardware) model, coil (hardware) model, etc.).

The power level prediction model determines a predicted power level for the implant sufficient to address the implant power needs. The predicted power level may include any indication of power for the implant (e.g., an actual amount of power, voltage, and/or electrical current; settings for providing the predicted power to the implant; etc.).

The power level prediction may be performed on an external device (e.g., external component 104, etc.) and/or on another computing system (e.g., computing device 110, etc.) in communication with the external device. In the case of the power level prediction being performed on the other computing system (e.g., to conserve processing and/or battery life, etc.), the other computing system may send the determined power level to the external device for providing power to the implant. The power level prediction model may be pre-trained with training data (e.g., from simulations as described below) on the external device, other computing system, and/or a separate system, and deployed for use on the external device and/or other computing system. Further, the power level prediction model may be dynamically or continuously updated or trained (and deployed) based on new information collected and obtained from the implant as described in more detail below.

The predicted power level and stimulation data produced from the audio signal processing path are provided to RF transceiver 122. The RF transceiver provides the corresponding power and stimulation data from sound processing unit 106 to implant 112 via wireless link 148.

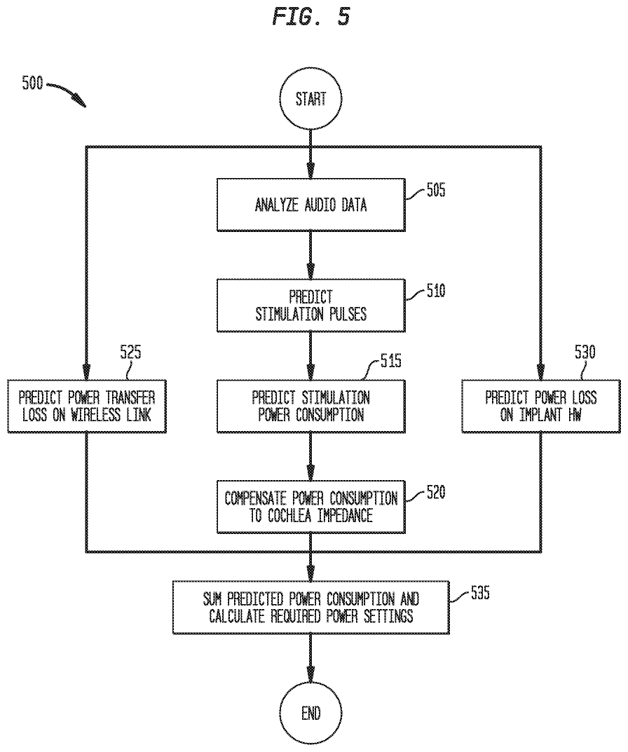

By leveraging artificial intelligence (AI) or machine learning, an external device is able to very accurately determine the power needs of an implant and accurately track the power needs over time. Referring to FIG. 4, a graph 400 illustrates, by way of example, a load 410 on an implant and predicted power or transferred power 420 for the implant. The graph plots load 410 and predicted power 420 along X and Y axes, where the X-axis represents time and the Y-axis represents an amount of power. As shown in FIG. 4, power 420 predicted by artificial intelligence (AI) or machine learning tracks or mirrors load 410 on the implant over time.

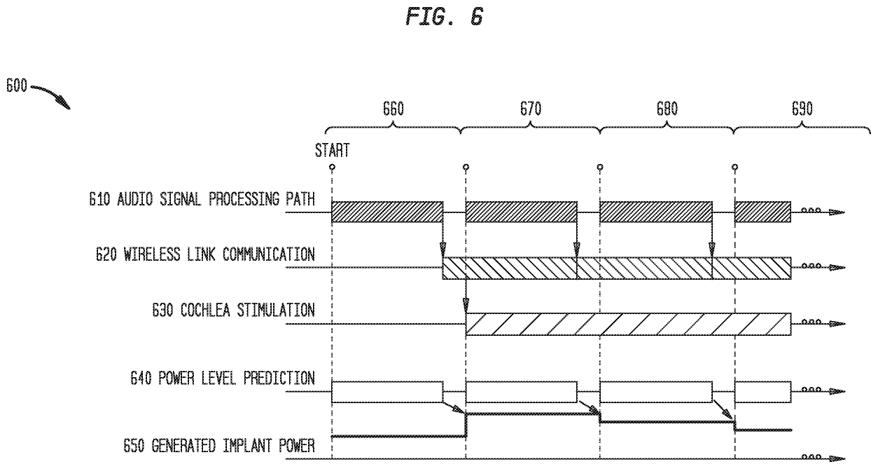

With reference now made to FIG. 5, depicted therein is a flowchart of a method 500 for determining a power level for an implantable device or implant using machine learning and based on audio signals according to certain embodiments. Method 500 may be performed by power level prediction logic 185 using power level prediction model 192, and may correspond to operation 315 of FIG. 3. The power level prediction model may include at least one machine learning model to perform one or more of the operations described below for FIG. 5. Method 500 may be performed to determine the power level independent of the audio signal processing path. Stated differently, method 500 determines the power level from the raw audio signals that are passed to the audio signal processing path. The predicted power level may include any indication of power for the implant (e.g., an actual amount of power, voltage and/or electrical current; settings for providing the predicted power to the implant, etc.). The power level prediction logic may receive various parameters for the power level prediction model to predict the power level for the implantable device. By way of example, the parameters may include audio input (e.g., in the time or frequency domain), electrical current of each stimulation pulse, power efficiency coefficients for the implant, audio signal processing path settings (e.g., coding type, maxima, comfort and threshold (C and T) levels, stimulation rate, etc.), audio classification (e.g., quiet or loud environment, audio characteristics, etc.) from an environment or audio classifier, RF link settings and/or characteristics (e.g., frame period, frame occupation, power transfer coefficient, amount of signal being enabled/transferred (e.g., more power may be transferred as more data is sent), etc.), cochlea impedance measurements, implant (hardware) model, and/or coil (hardware) model.

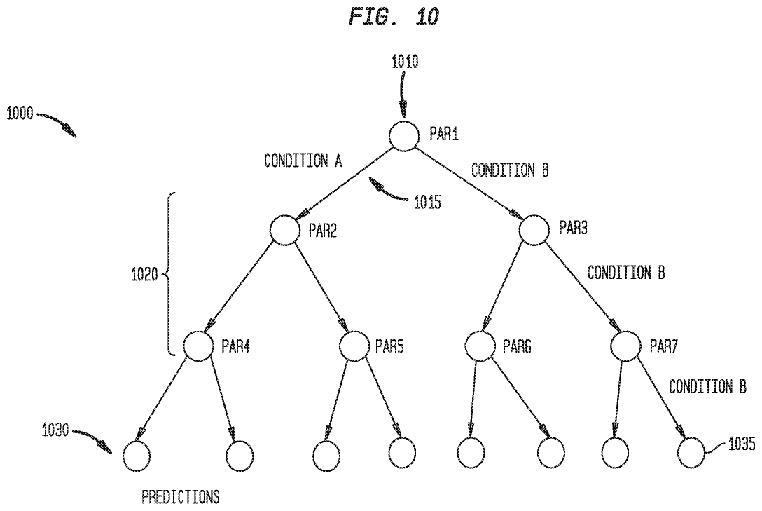

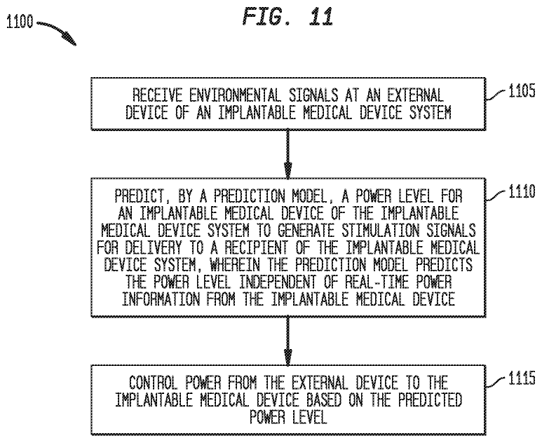

The audio data is received and analyzed by the power level prediction model at operation 505, and stimulation pulses are predicted at operation 510. The stimulation pulses may be predicted by performing the audio signal processing path 251 described above for FIG. 2 in accordance with the audio signal processing path settings (e.g., coding type, maxima, comfort and threshold (C and T) levels, stimulation rate, etc.). Alternatively, the stimulation pulses may be predicted by a stimulation machine learning model based on the audio signal processing path settings. The stimulation machine learning model may include any conventional or other machine learning models (e.g., mathematical/statistical, classifiers, decision tree, random forest, feed-forward, recurrent, convolutional, deep learning, or other neural networks, etc.) to predict the stimulation pulses. By of example, the stimulation machine learning model may include a neural network (FIG. 9) or a decision tree (FIG. 10), and may be trained with various audio signals and audio signal path settings as input, and corresponding stimulation pulses as known output in substantially the same manners described below.

Power consumption for the predicted stimulation pulses is determined at operation 515. The power consumption prediction may be determined based on attributes or characteristics of the predicted stimulation pulses (e.g., amplitude, frequency, electrical current, etc.). For example, the power consumption may be predicted via any conventional or other power prediction computations or techniques (e.g., formulas, relationships, etc.), or by a power consumption machine learning model based on the attributes or characteristics of the predicted stimulation pulses. The power consumption machine learning model may include any conventional or other machine learning models (e.g., mathematical/statistical, classifiers, decision tree, random forest, feed-forward, recurrent, convolutional, deep learning, or other neural networks, etc.) to predict the power consumption of the stimulation pulses. By of example, the power consumption machine learning model may include a neural network (FIG. 9) or a decision tree (FIG. 10), and may be trained with various stimulation pulses and associated characteristics (e.g., amplitude, frequency, electrical current, etc.) as input, and corresponding power consumption as known output in substantially the same manners described below.

The predicted power consumption may be adjusted to compensate for cochlea impedance at operation 520. The adjustment may be based on impedance data measurements. For example, the power consumption adjustment may be determined via any conventional or other computations or techniques (e.g., formulas, relationships, etc.), or by a compensation machine learning model based on the impedance data measurements. The compensation machine learning model may include any conventional or other machine learning models (e.g., mathematical/statistical, classifiers, decision tree, random forest, feed-forward, recurrent, convolutional, deep learning, or other neural networks, etc.) to adjust the predicted power consumption of the stimulation pulses. By of example, the compensation machine learning model may include a neural network (FIG. 9) or a decision tree (FIG. 10), and may be trained with various stimulation pules, characteristics, and power consumption as input, and corresponding adjusted power consumption as known output in substantially the same manners described below.

The power transfer loss of the wireless or inductive link is predicted at operation 525. The predicted power transfer loss may be based on a coil distance (or skin flap thickness of a user). For example, the power transfer loss may be determined via any conventional or other computations or techniques (e.g., formulas, relationships, etc.), or by a transfer loss machine learning model based on the coil distance (or skin flap thickness of a user). The transfer loss machine learning model may include any conventional or other machine learning models (e.g., mathematical/statistical, classifiers, decision tree, random forest, feed-forward, recurrent, convolutional, deep learning, or other neural networks, etc.) to predict the power transfer loss. By of example, the transfer loss machine learning model may include a neural network (FIG. 9) or a decision tree (FIG. 10), and may be trained with various characteristics of wireless links and coil distances as input, and corresponding power transfer loss as known output in substantially the same manners described below.

The power loss of the implant is predicted at operation 530. The predicted power loss of the implant may be based on the product model of the implant. For example, the power loss may be determined via any conventional or other techniques (e.g., formulas, relationships, specifications of the particular product model, etc.), or by a power loss machine learning model based on the product model specifications. The power loss machine learning model may include any conventional or other machine learning models (e.g., mathematical/statistical, classifiers, decision tree, random forest, feed-forward, recurrent, convolutional, deep learning, or other neural networks, etc.) to predict the power loss of the implant. By of example, the power loss machine learning model may include a neural network (FIG. 9) or a decision tree (FIG. 10), and may be trained with various product models and specifications as input, and corresponding power loss as known output in substantially the same manners described below.

The predicted power consumptions and power losses are combined (e.g., summed, weighted summation, etc.) to produce a power level (e.g., amount of power and/or power settings, etc.) for the implant. For example, the predicted power consumptions and power losses are combined (e.g., summed, weighted summation, etc.) to produce a resulting amount of power needed by the implant, and corresponding power settings for the wireless or inductive link are determined at operation 535 to provide the resulting amount of power to the implant. The wireless link settings control the amount of power that is sent to the implant, and can be the voltage level of the RF waveform. In case a packet protocol over the air is framed, the amount of RF energy within a frame can be an output setting. By way of example, the resulting amount of power may be determined via any conventional or other computations or techniques (e.g., formulas, relationships, etc.), while the corresponding settings may be determined based on the resulting amount of power (e.g., formulas, relationships, etc.) or by a mapping of the power settings to various power amounts.

Alternatively, the power level (e.g., amount of power and/or power settings, etc.) may be determined by an implant power machine learning model based on the predicted power consumptions and power losses. The implant power machine learning model may include any conventional or other machine learning models (e.g., mathematical/statistical, classifiers, decision tree, random forest, feed-forward, recurrent, convolutional, deep learning, or other neural networks, etc.) to determine the power level (e.g., amount of power and/or power settings, etc.). By of example, the implant power machine learning model may include a neural network (FIG. 9) or a decision tree (FIG. 10), and may be trained with various power consumptions and losses (and one or more of the input parameters) as input, and corresponding power levels (e.g., amounts of power and/or power settings) as known output in substantially the same manners described below.

In certain embodiments, the power level prediction model may employ an implant power prediction machine learning model that receives one or more of the input parameters, and produces the power level (e.g., resulting amount of power and/or power settings). The implant power prediction machine learning model produces the power level (e.g., resulting amount of power and/or power settings) based on the input parameters and information associated with the operations described above (e.g., operations 505-535). The input parameters may include audio input (e.g., in the time or frequency domain), electrical current of each stimulation pulse, power efficiency coefficients for the implant, signal processing path settings (e.g., coding type, maxima, comfort and threshold (C and T) levels, stimulation rate, etc.), audio classification (e.g., quiet or loud environment, audio characteristics, etc.) from an environment or audio classifier, RF link settings and/or characteristics (e.g., frame period, frame occupation, power transfer coefficient, amount of signal being enabled/transferred (e.g., more power may be transferred as more data is sent), etc.), cochlea impedance measurements, implant (hardware) model, and/or coil (hardware) model.

The implant power machine learning model may include any conventional or other machine learning models (e.g., mathematical/statistical, classifiers, decision tree, random forest, feed-forward, recurrent, convolutional, deep learning, or other neural networks, etc.) to determine the power level (e.g., power amount and/or power settings). By of example, the implant power machine learning model may include a neural network (FIG. 9) or a decision tree (FIG. 10), and may be trained with various sets of the input parameters as input, and corresponding power levels (e.g., power amounts and/or power settings) as known output in substantially the same manners described below.

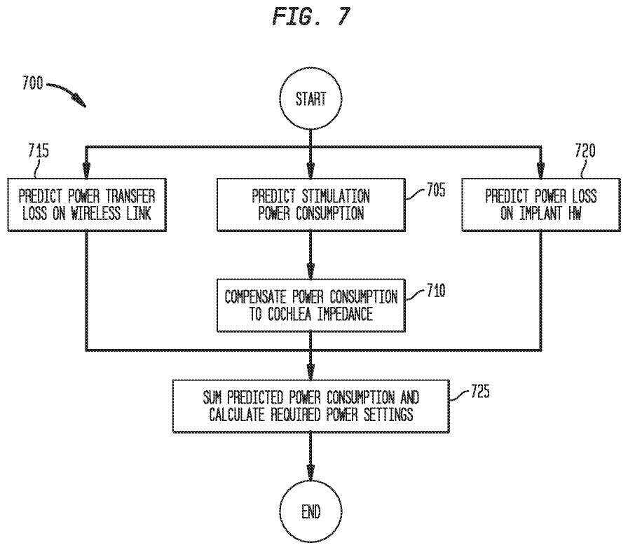

Referring to FIG. 6, an example timing diagram 600 is illustrated that depicts timing of processes and generated implant power within an implant system for controlling power to an implant. The power level is predicted independent of the audio signal processing path. Stated differently, the power level is determined from the raw audio signals that are passed to the audio signal processing path.

The processes include audio signal processing path 610 that generates output signals which can be used for delivering stimulation to a recipient as described herein, wireless link communication 620 between an external device and an implant, cochlea stimulation 630 performed by the implant, and power level prediction 640 performed by the external device and/or another computing system as described herein. Generated implant power 650 represents the predicted or generated power for the implant. The power level prediction determines the power level independent of the signal processing path. Stated differently, the power level is determined from the raw audio signals that are passed to the audio signal processing path. Timing diagram 600 illustrates, by way of example, a plurality of recurring time intervals 660, 670, 680, and 690.

Initially, audio signals are received and processed in time interval 660 by audio signal processing path 610 and power level prediction 640. An initial generated implant power 650 is provided for the implant. Audio signal processing path 610 processes the audio signals within time interval 660, and provides information concerning cochlea stimulation to wireless link communication 620 for transfer to the implant. The information is analyzed and corresponding cochlea stimulation 630 is initiated by the implant at the start of time interval 670.

Power level prediction 640 predicts the power level for the implant within time interval 660 based on the audio signals (e.g., independent of, and during processing of the audio signals by, audio signal processing path 610), and enables the corresponding power to be provided to the implant via wireless link communication 620. The generated implant power 650 is adjusted based on the predicted power level at the start of time interval 670.

New audio signals are received and processed in time interval 670 by audio signal processing path 610 and power level prediction 640. Audio signal processing path 610 processes the audio signals within time interval 670, and provides information concerning cochlea stimulation to wireless link communication 620 for transfer to the implant. The information is analyzed and corresponding cochlea stimulation 630 is provided by the implant at the start of time interval 680. Power level prediction 640 predicts the power level for the implant based on the new audio signals within time interval 670 (e.g., independent of, and during processing of the new audio signals by, audio signal processing path 610), and enables the corresponding power to be provided to the implant via wireless link communication 620. The generated implant power 650 is adjusted based on the predicted power at the start of time interval 680.

New audio signals are received at the start of subsequent time intervals (e.g., time interval 680, time interval 690, etc.), while the audio signals are processed to provide cochlear stimulation and adjust implant power in substantially the same manner described above for time interval 670.

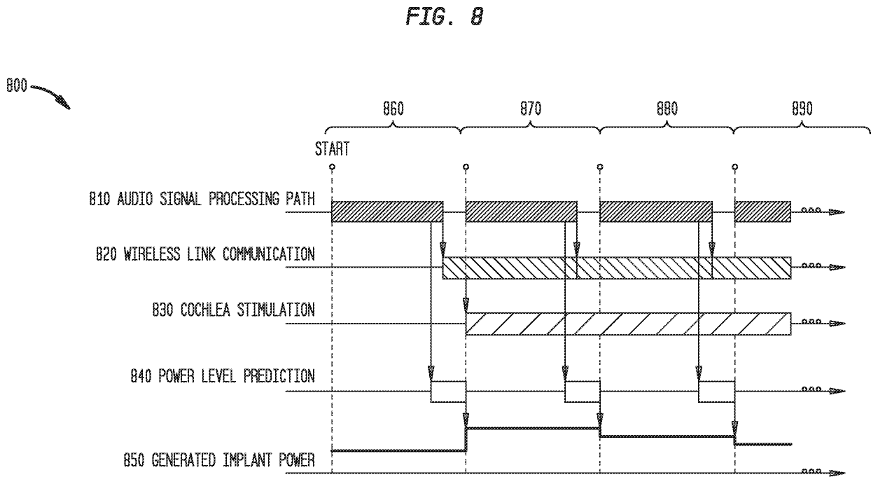

With reference now made to FIG. 7, depicted therein is a flowchart of a method 700 for determining power settings for an implantable component using machine learning and based on information from the audio signal processing path according to certain embodiments. Method 700 may be performed by power level prediction logic 185 using power level prediction model 192, and may correspond to operation 315 of FIG. 3. The power level prediction model may include at least one machine learning model to perform one or more of the operations described below for FIG. 7. Method 700 may be performed to determine the power level based on the audio signal processing path. Stated differently, method 700 determines the power level from information for the raw audio signals that are at least partially processed by the audio signal processing path (e.g., determined stimulation pulses, filter bins, module outputs 255, 257, 259, 261, and/or 263, etc.). The information may include any information obtained from any stage of the audio signal processing path (e.g., from pre-filterbank processing module 254, filterbank module 256, post-filterbank processing module 258, channel selection module 260, and/or mapping module 262). The predicted power level may include any indication of power for the implant (e.g., actual power, voltage, and/or electrical current; settings for providing the predicted power to the implant; etc.).

Initially, the power level prediction logic may receive various parameters for the power level prediction model to predict the power level for the implantable device. By way of example, the parameters may include audio input (e.g., in the time or frequency domain) or stimulation pulses, electrical current of each stimulation pulse, power efficiency coefficients for the implant, signal processing path settings (e.g., coding type, maxima, comfort and threshold (C and T) levels, stimulation rate, etc.), audio classification (e.g., quiet or loud environment, audio characteristics, etc.) from an environment or audio classifier, signal processing path information (e.g., filter bins or banks, signal strength levels, stimulation pulses, etc.), RF link settings and/or characteristics (e.g., frame period, frame occupation, power transfer coefficient, amount of signal being enabled/transferred (e.g., more power may be transferred as more data is sent), etc.), cochlea impedance measurements, implant (hardware) model, and/or coil (hardware) model.

The data from the audio signal processing path is received and analyzed, and may include stimulation pulses determined in accordance with the signal processing path settings (e.g., coding type, maxima, comfort and threshold (C and T) levels, stimulation rate, etc.). Power consumption for the stimulation pulses is determined at operation 705. The power consumption prediction may be determined based on attributes or characteristics of the stimulation pulses (e.g., amplitude, frequency, electrical current, etc.). For example, the power consumption may be predicted via any conventional or other computations or techniques (e.g., formulas, relationships, etc.), or by the power consumption machine learning model based on the attributes or characteristics of the stimulation pulses in substantially the same manner described above.

The predicted power consumption may be adjusted to compensate for cochlea impedance at operation 710. The adjustment may be based on impedance data measurements. For example, the power consumption adjustment may be determined via any conventional or other computations or techniques (e.g., formulas, relationships, etc.), or by the compensation machine learning model based on the impedance data measurements in substantially the same manner described above.

The power transfer loss of the wireless or inductive link is predicted at operation 715. The predicted power transfer loss may be based on a coil distance (or skin flap thickness of a user). For example, the power transfer loss may be determined via any conventional or other computations or techniques (e.g., formulas, relationships, etc.), or by the transfer loss machine learning model based on the coil distance (or skin flap thickness of a user) in substantially the same manner described above.

The power loss of the implant is predicted at operation 720. The predicted power loss of the implant may be based on the product model of the implant. For example, the power loss may be determined via any conventional or other computations or techniques (e.g., formulas, relationships, etc.), or by the power loss machine learning model based on the product model specifications in substantially the same manner described above.

The predicted power consumptions and power losses are combined (e.g., summed, weighted summation, etc.) to produce a power level (e.g., amount of power and/or power settings, etc.) for the implant. For example, the predicted power consumptions and power losses are combined (e.g., summed, weighted summation, etc.) to produce a resulting amount of power needed by the implant, and corresponding power settings for the wireless or inductive link are determined at operation 725 to provide the resulting amount of power to the implant. The wireless link settings control the amount of power that is sent to the implant, and can be the voltage level of the RF waveform. In case a packet protocol over the air is framed, the amount of RF energy within a frame can be an output setting. By way of example, the resulting amount of power may be determined via any conventional or other computations or techniques (e.g., formulas, relationships, etc.), while the corresponding settings may be determined based on the resulting amount of power (e.g., formulas, relationships, etc.) or by a mapping of the power settings to various power amounts.

Alternatively, the power level (e.g., amount of power and/or power settings) may be determined by the implant power machine learning model based on the predicted power consumptions and power losses in substantially the same manner described above.

In certain embodiments, the power level prediction model may employ the implant power prediction machine learning model that receives the audio signals and information from the audio signal processing path (and, optionally, one or more other ones of the input parameters), and produces the power level (e.g., resulting amount of power and/or power settings). The implant power prediction machine learning model produces the power level (e.g., resulting amount of power and/or power settings) based on the received audio signals, information, and other parameters and information associated with the operations described above (e.g., operations 705-725). The input parameters may include audio input (e.g., in the time or frequency domain) or stimulation pulses, electrical current of each stimulation pulse, power efficiency coefficients for the implant, signal processing path settings (e.g., coding type, maxima, comfort and threshold (C and T) levels, stimulation rate, etc.), audio classification (e.g., quiet or loud environment, audio characteristics, etc.) from an environment or audio classifier, signal processing path information (e.g., filter bins or banks, signal strength levels, stimulation pulses, etc.), RF link settings and/or characteristics (e.g., frame period, frame occupation, power transfer coefficient, amount of signal being enabled/transferred (e.g., more power may be transferred as more data is sent), etc.), cochlea impedance measurements, implant (hardware) model, and/or coil (hardware) model.

The implant power machine learning model may include any conventional or other machine learning models (e.g., mathematical/statistical, classifiers, decision tree, random forest, feed-forward, recurrent, convolutional, deep learning, or other neural networks, etc.) as described above to determine the power level (e.g., resulting power level and/or power settings). By of example, the implant power machine learning model may include a neural network (FIG. 9) or a decision tree (FIG. 10), and may be trained with the audio signals and information from the audio signal processing path (and, optionally, one or more other ones of the input parameters) as input, and corresponding power levels (e.g., power amounts and/or power settings) as known output in substantially the same manners described below.

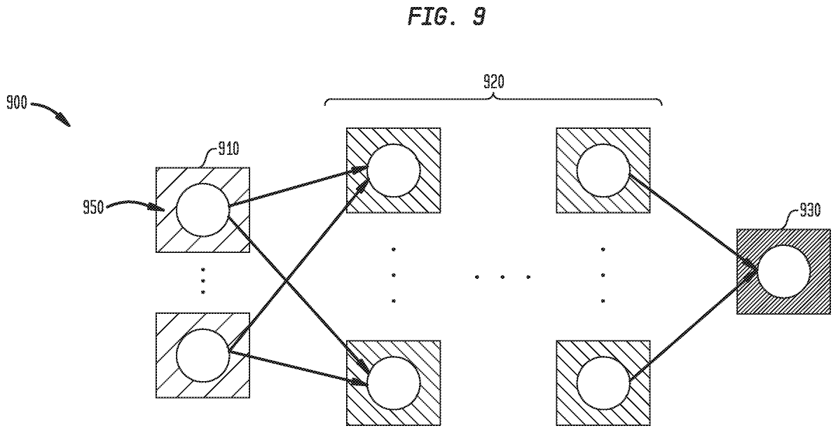

Referring to FIG. 8, an example timing diagram 800 is illustrated that depicts timing of processes and generated implant power within an implant system for controlling power to an implant. The power is predicted based on the audio signal processing path. Stated differently, the power is determined from the processed audio signals from the audio signal processing path.

The processes include audio signal processing path 610 that generates output signals which can be used for delivering stimulation to a recipient as described herein, wireless link communication 620 between an external device and an implant, cochlea stimulation 630 performed by the implant, and power level prediction 640 performed by the external device and/or another computing system as described herein. Generated implant power 650 represents the predicted or generated power for the implant. The power level prediction determines the power based on information from the audio signal processing path. Stated differently, the power is determined from information for the audio signals that are processed by the audio signal processing path. The information may include any information obtained from any stage of the audio signal processing path (e.g., from pre-filterbank processing module 254, filterbank module 256, post-filterbank processing module 258, channel selection module 260, and/or mapping module 262). Timing diagram 800 illustrates, by way of example, a plurality of recurring time intervals 860, 870, 880, and 890.

Initially, audio signals are received and processed in time interval 860 by audio signal processing path 610. An initial generated implant power 650 is provided for the implant. Audio signal processing path 610 processes the audio signals within time interval 860, and provides information concerning cochlea stimulation to wireless link communication 620 for transfer to the implant. The information is analyzed and corresponding cochlea stimulation 630 is initiated by the implant at the start of time interval 870.

Power level prediction 640 receives information from various stages of audio signal processing path 610 within time interval 860, and predicts the power for the implant based on the information from the audio signal processing path (e.g., determined stimulation pulses, filter bins, module outputs 255, 257, 259, 261, and/or 263, etc.). The power level prediction enables the corresponding power to be provided to the implant via wireless link communication 620. The generated implant power 650 is adjusted based on the predicted power at the start of time interval 870.

New audio signals are received and processed in time interval 870 by audio signal processing path 610. Audio signal processing path 610 processes the audio signals within time interval 870, and provides information concerning cochlea stimulation to wireless link communication 620 for transfer to the implant. The information is analyzed and corresponding cochlea stimulation 630 is provided by the implant at the start of time interval 880. Power level prediction 640 receives information from various stages of the audio signal processing path within time interval 870 and predicts the power for the implant based on the information (e.g., determined stimulation pulses, filter bins, module outputs 255, 257, 259, 261, and/or 263, etc.). The power level prediction enables the corresponding power to be provided to the implant via wireless link communication 620. The generated implant power 650 is adjusted based on the predicted power at the start of time interval 880.

New audio signals are received at the start of subsequent time intervals (e.g., time interval 880, time interval 890, etc.), while the audio signals are processed to provide cochlear stimulation and adjust implant power in substantially the same manner described above for time interval 870.

The power level prediction model may employ various machine learning models as described above (e.g., stimulation machine learning model, power consumption machine learning model, compensation machine learning model, transfer loss machine learning model, power loss machine learning model, implant power machine learning model, implant power prediction machine learning model, etc.). These machine learning models may be implemented by any conventional or other machine learning models (e.g., mathematical/statistical, classifiers, decision tree, random forest, feed-forward, recurrent, convolutional, deep learning, or other neural networks, etc.).

By way of example, one or more of the machine learning models of the power level prediction model (e.g., stimulation machine learning model, power consumption machine learning model, compensation machine learning model, transfer loss machine learning model, power loss machine learning model, implant power machine learning model, implant power prediction machine learning model, etc.) may employ a neural network. An example neural network 900 is illustrated in FIG. 9. Neural network 900 may include an input layer 910, one or more intermediate layers 920 (e.g., including any hidden layers), and an output layer 930. Each layer includes one or more neurons 950, where the input layer neurons receive input associated with a particular machine learning model of the power level prediction model as described above (e.g., audio signals, one or more of the other input parameters, etc.), and may be associated with weight values. The neurons of the intermediate and output layers are connected to one or more neurons of a preceding layer, and receive as input the output of a connected neuron of the preceding layer. Each connection is associated with a weight value, and each neuron produces an output based on a weighted combination of the inputs to that neuron. The output of a neuron may further be based on a bias value for certain types of neural networks (e.g., recurrent types of neural networks, etc.).

The weight (and bias) values may be adjusted based on various training techniques. For example, the machine learning of the neural network may be performed using a training set of data as input and corresponding known outputs for the particular machine learning model of the power level prediction model being trained as described above, where the neural network attempts to produce the provided output and uses an error from the output (e.g., difference between produced and known outputs) to adjust weight (and bias) values (e.g., via backpropagation or other training techniques).

In an embodiment, feature vectors may be extracted from the training set input data for the particular machine learning model of the power level prediction model and used for the training as input, while their known corresponding outputs may be used for the training as known output. A feature vector may include any suitable features of the training set input data. For example, features of audio signals may include fundamental or other frequency, pitch, amplitude or intensity, etc.

The output layer of the neural network indicates the resulting output for input data for the particular machine learning model of the power level prediction model. The output layer neurons may further indicate a probability for the resulting output.

By way of further example, one or more of the machine learning models of the power level prediction model (e.g., stimulation machine learning model, power consumption machine learning model, compensation machine learning model, transfer loss machine learning model, power loss machine learning model, implant power machine learning model, implant power prediction machine learning model, etc.) may employ a machine learning decision tree. An example decision tree 1000 is illustrated in FIG. 10. Decision tree 1000 includes a root node 1010 representing a total population (e.g., the entire set of parameters/inputs and outputs for the particular machine learning model of the power level prediction model), one or more decision nodes 1020, and one or more leaf nodes 1030 each associated with an output for the particular machine learning model of the power level prediction model. The decision tree starts from root node 1010, and examines each of the parameters/inputs in order to partition the total population into smaller groups (e.g., based on values of that parameter/input). The parameter/input that provides the groups with the most similar members (e.g., having the values for the other parameters and/or output) is utilized as a decision point, where a certain value of the parameter/input used to form the groups is used as a condition for edges 1015 for traversing paths extending from the root node to child nodes 1020. The analysis is repeated for the smaller groups formed by the child nodes (with the remaining parameters/inputs) to extend the decision tree with additional decision nodes 1020 and edges 1015 until paths through the decision tree terminate at leaf nodes 1030 representing the predicted output for the particular machine learning model of the power level prediction model. The decision tree may be tested on a sample of a data set to ensure predicted outputs are accurate. Each path of the resulting decision tree leading to a leaf node or predicted output includes the parameters/input (and corresponding values or conditions) that provide the scenario for the corresponding output.

By way of example, root node 1010 may be associated with a parameter (e.g., PAR1 as viewed in FIG. 10) and edges 1015 indicating conditions for PAR1 (e.g., CONDITION A, CONDITION B as viewed in FIG. 10) leading to child nodes 1020 associated with other parameters (e.g., PAR2 and PAR3 as shown in FIG. 10). The conditions may pertain to values for PAR1. Decision nodes 1020 for PAR2 and PAR3 may be associated with corresponding edges 1015 indicating conditions for those parameters and leading to child nodes associated with other parameters (e.g., PAR4, PAR5, PAR6, and PAR7 as shown in FIG. 10). The conditions may pertain to values for PAR2 and PAR3. Decision nodes 1020 for PAR4, PAR5, PAR6, and PAR7 may be associated with corresponding edges 1015 indicating conditions for those parameters leading to leaf nodes 1030 representing the desired output for the particular machine learning model of the power level prediction model. The conditions may pertain to values for PAR4, PAR5, PAR6, and PAR7.

Decision tree 1000 is constructed to produce predicted outputs for the particular machine learning model of the power level prediction model using the parameters/input for the particular machine learning model. For example, a parameter/input (PAR1) may partition the data set into a first group having certain values for the parameter/input, and may serve as a root node. Another parameter/input (PAR3) may further partition this first group into a second group having certain values for the parameter/input (PAR3), and serve as a decision node 1020 (a child of the root node). Yet another parameter/input (PAR7) may further partition the second group into a third group having certain values for the parameter/input (PAR7), and serve as a decision node 1020 (a child of the child node). The decision tree partitions the groups based on the parameters/input until the leaf nodes 1030 include a group of scenarios having the same values for parameters/input that produce the corresponding output for the particular machine learning model of the power level prediction model.

The parameters/input for the particular machine learning model of the power level prediction model are applied to decision tree 1000 to determine a path to a leaf node 1030 indicating a predicted output for the particular machine learning model, where branches at the nodes are traversed based on the values of the corresponding parameters/input satisfying the edge conditions. The resulting path indicates the values of the corresponding parameters/input associated with the predicted output. By way of example, a predicted output associated with a leaf node 1035 may be produced in response to PAR1, PAR3, and PAR7 each satisfying CONDITION B of corresponding edges 1015 along a path from root node 1010 to leaf node 1035.

The decision tree may be constructed/trained, and/or updated (e.g., the attributes, conditions, and/or outputs) using any conventional or other metrics or techniques, such as entropy (e.g., randomness or uncertainty of data), Gini index (e.g., misclassification of a random data point), information gain (e.g., reduction in entropy or Gini index due to a split or decision), iterative dichotomiser 3(ID3 ), C4.5, classification and regression trees (CART), chi-square automatic interaction detector (CHAID), multivariate adaptive regression splines (MARS), etc. The decision tree may be trained on new data or feedback data (e.g., actual power measurements) by determining the attributes and conditions until a metric is within a desired tolerance or range (e.g., entropy, Gini index, and/or information gain).

Power level prediction model 192 may be trained in various manners. The power level prediction model may be pre-trained with training data (e.g., predetermined training data, data from simulations or actual hardware, etc.) on the external device, other computing system, and/or a separate system, and deployed for use on the external device and/or other computing system as described below. Further, the power level prediction model may be dynamically or continuously updated or trained (and deployed) based on new information collected from the implant as described below. The training may be performed by power level prediction logic 185. The machine learning models of the power level prediction model (e.g., stimulation machine learning model, power consumption machine learning model, compensation machine learning model, transfer loss machine learning model, power loss machine learning model, implant power machine learning model, implant power prediction machine learning model, etc.) may be trained together to train the power level prediction model to produce the desired output (e.g., based on a set of input and known output for the power level prediction model), and/or the machine learning models of the power level prediction model may be trained individually to produce their corresponding predicted outputs (e.g., stimulation pulses, power consumption, compensated power consumption, power transfer loss, implant power, power level, etc.).

In some instances, the power level prediction model and/or corresponding individual machine learning models (e.g., stimulation machine learning model, power consumption machine learning model, compensation machine learning model, transfer loss machine learning model, power loss machine learning model, implant power machine learning model, implant power prediction machine learning model, etc.) may be trained using computerized or other simulations. The power level prediction model is run in a simulator which contains a complex model for the wireless link, implant hardware, and cochlea. An audio scenario with corresponding input parameters is presented to the power level prediction model that produces the wireless link settings to be applied. This information is fed into the simulated system model that calculates the actual power consumed and the power received based on the settings from the power level prediction model. The actual power, received power, and information from the simulation can be used for training sets to adjust the behavior of the power level prediction model and/or the corresponding individual machine learning models. For example, a neural network of the power level prediction model and/or the corresponding individual machine learning models may be trained from this data by using an error from the output (e.g., difference between produced and known outputs) to adjust weight (and bias) values (e.g., via backpropagation or other training techniques) in substantially the same manner described above. A decision tree of the power level prediction model and/or the corresponding individual machine learning models may be trained from this data by determining the attributes and conditions until a metric is within a desired tolerance or range (e.g., entropy, Gini index, and/or information gain) in substantially the same manner described above.

In some instances, the simulated system may be built using real hardware. In this case, the behavior of the power level prediction model and/or the corresponding individual machine learning models (e.g., neural network, decision tree, etc.) may be adjusted based on logged data produced from the hardware system in substantially the same manner described above.

In some embodiments, the power level prediction model and/or the corresponding individual machine learning models may be trained using various training data. For example, the training data may include a wide variety of audio files which provide the power level prediction model and/or the corresponding individual machine learning models with various different scenarios. The scenarios may range from quiet scenarios to very loud scenarios, such as music concerts. Further, the scenarios may include real world examples, such as walking in a park of a city. These various scenarios may be used as training data to train the power level prediction model and/or the corresponding individual machine learning models to correctly predict their corresponding outputs in each of the use cases (e.g., stimulation pulses, power consumption, compensated power consumption, power transfer loss, implant power, power level, etc.).

Moreover, a very wide range of fitting parameters that influence the audio signal processing path may be used for training the power level prediction model and/or the corresponding individual machine learning models. This data can be based on a statical set of parameters extracted from knowledge in the field, and/or all the possible combinations can be calculated and used for training.

In addition, statical data of impedance variation of the cochlea over a day or other time interval and/or variations in skin flap over a lifetime of the product or implant may be used for training. This enables the power level prediction model and/or the corresponding individual machine learning models to learn to compensate for these types of variations.

By way of example, a neural network of the power level prediction model and/or the corresponding individual machine learning models may be trained from these training data by using an error from the output (e.g., difference between produced and known outputs) to adjust weight (and bias) values (e.g., via backpropagation or other training techniques) in substantially the same manner described above. A decision tree of the power level prediction model and/or the corresponding individual machine learning models may be trained from these training data by determining the attributes and conditions until a metric is within a desired tolerance or range (e.g., entropy, Gini index, and/or information gain) in substantially the same manner described above.

The power level prediction model and/or the corresponding individual machine learning models (e.g., neural network, decision tree, etc.) may be trained using an entirety or any portion of the training data in substantially the same manner described above.

In some embodiments, the power level prediction model and/or the corresponding individual machine learning models may be dynamically or continuously trained using information from the implant that is returned at a low, non-operational critical rate. This information is used to tune the power level prediction model and/or the corresponding individual machine learning models to improve implant power consumption prediction, and/or to fine tune/adjust the power level prediction model and/or the corresponding individual machine learning models to slow varying parameters, such as cochlea impedances and skin flap.

In some instances, implant power level and corresponding data is returned with a timestamp of when an event was recorded. The training can correlate this data to historical calculations and verify that the historical calculations are correct. In case an improvement is detected, the power level prediction model and/or the corresponding individual machine learning models are tuned based on the data from the implant to produce better predictions. The feedback data from the implant may include a power level measurement, out of compliance events (e.g., indicating that there is not enough power being sent but the implant did not yet reset), out of power reset events on the implant, impedance measurements, and/or other information associated with an event. For example, a neural network of the power level prediction model and/or the corresponding individual machine learning models may be trained from this data by using an error from the output (e.g., difference between produced and known outputs) to adjust weight (and bias) values (e.g., via backpropagation or other training techniques) in substantially the same manner described above. A decision tree of the power level prediction model and/or the corresponding individual machine learning models may be trained from this data by determining the attributes and conditions until a metric is within a desired tolerance or range (e.g., entropy, Gini index, and/or information gain) in substantially the same manner described above.

The power level prediction model and/or the corresponding individual machine learning models (e.g., neural network, decision tree, etc.) may be trained using an entirety or any portion of the feedback data in substantially the same manner described above.

In some instances, the power level prediction model and/or the corresponding individual machine learning models may be trained using feedback data on a reset event. This mechanism is substantially similar to the training based on feedback data described above, but depends less on the active back link. When an error case of no (or insufficient) power is detected, the implant system logs the event and corresponding information in non-volatile memory. This data can be read by the external device (e.g., external component 104, etc.) or other computing system (e.g., computing device 110), and can be used to train the power level prediction model and/or the corresponding individual machine learning models on the external device (or other computing system and deployed to the external device). The events and corresponding information may include a power level measurement, out of compliance events (e.g., indicating that there is not enough power being sent but the implant did not yet reset), out of power reset events on the implant, impedance measurements, etc. For example, a neural network of the power level prediction model and/or the corresponding individual machine learning models may be trained from this data by using an error from the output (e.g., difference between produced and known outputs) to adjust weight (and bias) values (e.g., via backpropagation or other training techniques) in substantially the same manner described above. A decision tree of the power level prediction model and/or the corresponding individual machine learning models may be trained from this data by determining the attributes and conditions until a metric is within a desired tolerance or range (e.g., entropy, Gini index, and/or information gain) in substantially the same manner described above.

The power level prediction model and/or the corresponding individual machine learning models (e.g., neural network, decision tree, etc.) may be trained using an entirety or any portion of the training data in substantially the same manner described above.

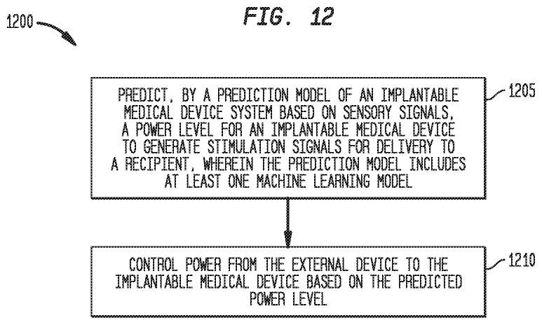

With reference now made to FIG. 11, depicted therein is a flowchart of a method 1100 for implementing the techniques of the present disclosure. Method 1100 begins in operation at 1105, which can include receiving sensory/environmental signals (e.g., light signals, audio signals, etc.) by an external device of an implantable medical device system. At 1110, the method includes predicting, by a prediction model (e.g., of the external device or another connected device), a power level for an implantable medical device of the implantable medical device system to generate stimulation signals for delivery to a recipient of the implantable medical device system. The prediction model predicts the power level independent of real-time power information for the stimulation from the implantable medical device and, in certain examples, based on the environmental signals. At 1115, the method includes controlling power from the external device to the implantable medical device based on the predicted power level. Accordingly, the method of flowchart 1100 provides for a process in which a power level may be determined and controlled by an external component to an implantable component of a medical device system independent of power information from the implantable component.

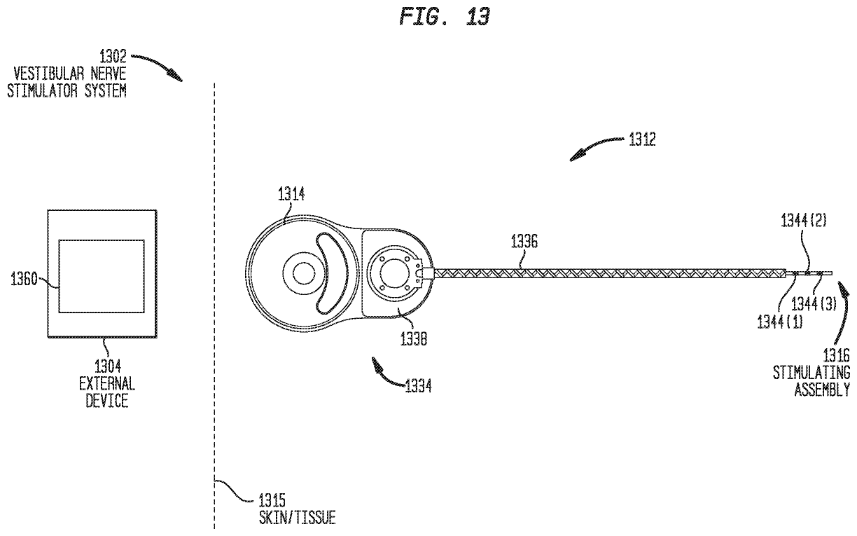

With reference now made to FIG. 12, depicted therein is a flowchart of a method 1200 for implementing the techniques of the present disclosure. Method 1200 begins in operation at 1205, which can include predicting, by a prediction model of an external device of an implantable medical device system based on sensory/environmental (e.g., audio, light, etc.) signals, a power level for an implantable medical device to generate stimulation signals for stimulation of a recipient. The prediction model includes at least one machine learning model. At 1210, the method can include controlling power from the external device to the implantable medical device for the stimulation based on the predicted power level. Accordingly, the method of flowchart 1200 provides for a process in which power may be determined and controlled by an external component to an implantable component of a medical device system based on artificial intelligence (AI) or machine learning.

As previously described, the technology disclosed herein can be applied in any of a variety of circumstances and with a variety of different devices. One example device that can benefit from technology disclosed herein is described in more detail in FIG. 13. In particular, FIG. 13 illustrates an example vestibular nerve stimulator system 1302, with which embodiments presented herein can be implemented. As shown, the vestibular nerve stimulator system 1302 comprises an implantable component (vestibular stimulator) 1312 and an external device/component 1304 (e.g., external processing device, battery charger, remote control, etc.). The external device 1304 comprises a transceiver unit 1360. As such, the external device 1304 is configured to transfer data (and potentially power) to the vestibular stimulator 1312.

The vestibular stimulator 1312 comprises an implant body (main module) 1334, a lead region 1336, and a stimulating assembly 1316, all configured to be implanted under the skin/tissue (tissue) 1315 of the recipient. The implant body 1334 generally comprises a hermetically-sealed housing 1338 in which RF interface circuitry, one or more rechargeable batteries, one or more processors, and a stimulator unit are disposed. The implant body 1334 also includes an internal/implantable coil 1314 that is generally external to the housing 1338, but which is connected to the transceiver via a hermetic feedthrough (not shown).

The stimulating assembly 1316 comprises a plurality of electrodes 1344 disposed in a carrier member (e.g., a flexible silicone body). In this specific example, the stimulating assembly 1316 comprises three (3) stimulation electrodes, referred to as stimulation electrodes 1344(1), 1344(2), and 1344(3). The stimulation electrodes 1344(1), 1344(2), and 1344(3) function as an electrical interface for delivery of electrical stimulation signals to the recipient's vestibular system.

The stimulating assembly 1316 is configured such that a surgeon can implant the stimulating assembly adjacent the recipient's otolith organs via, for example, the recipient's oval window. It is to be appreciated that this specific embodiment with three stimulation electrodes is merely illustrative and that the techniques presented herein may be used with stimulating assemblies having different numbers of stimulation electrodes, stimulating assemblies having different lengths, etc.

In operation, the external device 1304, and/or another external device, can be configured to implement the techniques presented herein. That is, the external device 1304 and/or another external device, can determine and control the optimal amount of power to the implantable component (vestibular simulator 1312), as described elsewhere herein.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments can be combined with another in any of a number of different manners.

1. A method, comprising:
receiving environmental signals at an external device of an implantable medical device system;
predicting, by a prediction model, a power level for an implantable medical device of the implantable medical device system to generate stimulation signals for delivery to a recipient of the implantable medical device system based on the environmental signals, wherein the prediction model predicts the power level based independent of real-time power information from the implantable medical device; and
controlling power transmitted from the external device to the implantable medical device based on the predicted power level.

2. The method of claim 1, wherein the prediction model includes at least one machine learning model.

3. The method of claim 1, wherein the prediction model predicts the power level further based on the environmental signals.

4. The method of claim 3, wherein the prediction model predicts the power level further based on settings for signal processing of the environmental signals to produce the stimulation signals.

5. (canceled)

6. The method of claim 1, wherein the prediction model predicts the power level based on settings of a wireless link between the external device and the implantable medical device.

7. The method of claim 1, wherein the prediction model predicts the power level based on one or more electrode impedance measurements associated with the implantable medical device.

8. The method of claim 1, wherein the prediction model predicts the power level based on a product model of the implantable medical device or based on a product model of a coil used for providing a link between the external device and the implantable medical device.

9. (canceled)

10. (canceled)

11. The method of claim 1, wherein the power level indicates an attribute of a signal sent over a wireless link between the external device and the implantable medical device.

12. (canceled)

13. (canceled)

14. The method of claim 1, wherein a wireless link between the external device and the implantable medical device uses a framed packet protocol, and wherein the power level indicates an amount of energy for a signal of the wireless link within a frame.

15. (canceled)

16. The method of claim 1, wherein the environmental signals are audio signals, and wherein the method comprises:
converting, with an audio signal processing path, the audio signals to output signals,
wherein the prediction model predicts the power level further based on information associated with the audio signal processing path.

17. (canceled)

18. (canceled)

19. The method of claim 2, further comprising:
training the at least one machine learning model based on power consumption of the implantable medical device determined by a computerized simulation of the implantable medical device, a link to the external device, and attributes of a recipient.

20. The method of claim 2, further comprising:
training the at least one machine learning model based on power consumption of the implantable medical device determined by monitoring a hardware implementation of the implantable medical device and a link to the external device.

21. The method of claim 2, further comprising:
training the at least one machine learning model based on feedback data from the implantable medical device.

22. The method of claim 21, wherein the feedback data includes one or more from a group of: a power level measurement, an out of compliance event, an out of power reset event, and impedance measurements.

23-35. (canceled)

36. An external device of an implantable medical device system comprising:
memory for storing data; and
one or more processors, wherein the one or more processors are configured to:
predict, by a prediction model based on sensory signals, a power level for an implantable medical device to generate stimulation signals for delivery to a recipient, wherein the prediction model includes at least one machine learning model; and
control power to the implantable medical device for the stimulation based on the predicted power level.

37. The external device of claim 36, wherein the prediction model predicts the power level further based on one or more from a group of: settings for signal processing of the sensory signals to produce the stimulation signals, settings of a wireless link to the implantable medical device, impedance measurements of a recipient cochlea, a product model of the implantable medical device, and a product model of a coil used for providing a link to the implantable medical device.

38. The external device of claim 36, wherein the power level indicates settings for a wireless link to provide the power to the implantable medical device.

39. The external device of claim 36, wherein the power level indicates a voltage of an RF signal sent over a wireless link to the implantable medical device.

40. The external device of claim 36, wherein a wireless link to the implantable medical device uses a framed packet protocol, and wherein the power level indicates an amount of RF energy for a signal of the wireless link within a frame.

41. The external device of claim 36, wherein the sensory signals comprise audio signals that are at least partially processed by an audio signal processing path processing the audio signals to produce the stimulation signals, and wherein the prediction model predicts the power level further based on information from the audio signal processing path.

42-53. (canceled)
